(19) United States
(12) Patent Application Publication
(10) Pub. No.: US 2007/0027369 A1
(43) Pub. Date: Feb. 1, 2007

(54) Apparatus and methods for assessing human physical performance

(76) Inventors: Guido Pagnacco, Cheyenne, WY (US); Elena Oggero, Cheyenne, WY (US); A. Bob Henderson, Marion, IL (US)

Correspondence Address: CAHN & SAMUELS LLP, 2000 P STREET NW, SUITE 200, WASHINGTON, DC, 20036, US

(21) Appl. No.: 11/191,728

(22) Filed: Jul. 28, 2005

(51) Int. Cl. A61B 5/00; A61B 5/02; A61B 13/00; A61B 5/04; A61B 5/103

(52) U.S. Cl. 600/301; 600/546; 600/587; 600/485; 600/481; 600/555; 600/558; 600/559

(57) ABSTRACT

An apparatus for assessing human physical performance includes one or more force measuring devices; one or more instruments for measuring at least one of kinesthetic, vestibular, visual, auditory, somatosensory, or cardiovascular information; a control apparatus; a computing apparatus; a storage apparatus; an input apparatus; and an output apparatus. The apparatus may also include one or more instruments for measuring at least one of vestibular, visual, or somatosensory information. In addition, methods may be used to assess human physical performance by assessing one or more of: (1) muscular performance; (2) postural balance/equilibrium and nervous system processing of kinesthetic information; (3) postural balance/equilibrium and nervous system processing of at least one of vestibular, visual, auditory, or somatosensory information; (4) postural balance/equilibrium and at least one other physiological parameter; or (5) neuromuscular abilities of a person during a movement requiring power followed by a stabilization period.

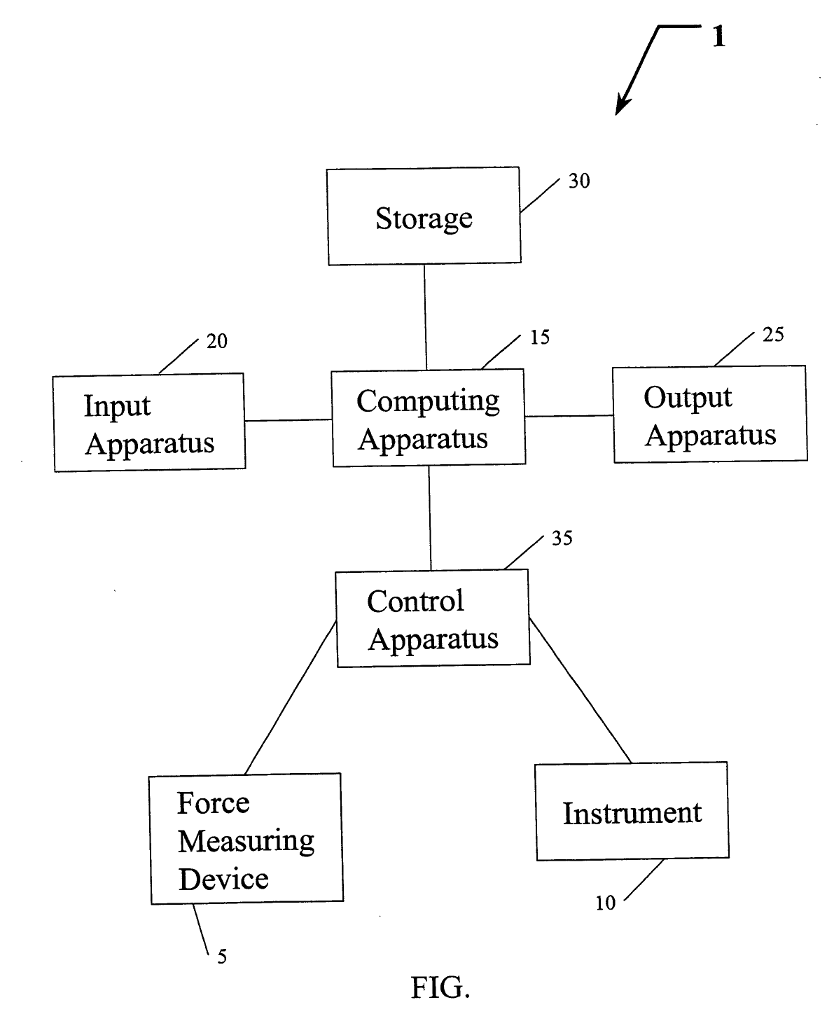

### FIELD OF INVENTION

The present invention is directed to an apparatus and methods for assessing human physical performance and for ascertaining the general neuromuscular health of a person.

### BACKGROUND OF INVENTION

With the competitiveness of today's lifestyle and increased life expectancy, there is a much greater interest in evaluating human physical performance at all levels, involving many medical fields, ranging from sports medicine to physical rehabilitation and from pediatrics to geriatrics.

Depending on the age group involved, different aspects of human physical performance may be assessed. For example, in younger populations, there is an increasing interest in evaluating and enhancing muscular performance. In elderly populations, there is an increasing interest in evaluating and improving postural balance and equilibrium. In all age populations, there is an increasing need for appropriate and effective physical rehabilitation after injuries or surgeries.

Whatever the reason, the assessment of human physical performance may be divided into three groups.

A first group for assessing human physical performance comprises the measurement of one or more of force, mechanical power, mechanical energy, acceleration, velocity, or distance traveled during specific movements of a person. This measurement is important to assess human physical performance, in particular muscular strength and performance. In sports training and sports medicine it is important to determine the proper training for an athlete, as many sports require athletes to be trained for maximum power. Other sports require training for maximum force, also known as resistance training. Sprinters, tennis players, basketball players, or high jumpers are trained for power. However, most football players, especially those in defensive roles, are trained for maximal force. This measurement is also important in physical therapy and other clinical settings as it can be used to monitor recovery after injury and to detect and quantify pathological conditions that affect muscular strength, such as distonia, Parkinson's disease, stroke, and multiple sclerosis.

A second group for assessing human physical performance comprises the measurement of postural balance and equilibrium by investigating how information received by the vestibular, visual, auditory, kinesthetic, or somatosensory systems is integrated into and acted upon by the nervous system. The existence of one or more of several different medical conditions can cause part of the information that is relayed to the nervous system to be missing, to be incorrect, or to be incorrectly processed by the nervous system, thereby resulting in a balance problem. Balance problems may be manifested in any of several different ways, for example, dizziness, an increased risk of falling, reduced reaction time, diminished physical ability, and the like. It thus becomes important to determine which sensory receptor or pathway is not sending the proper information to or from the nervous system, or why the nervous system is not processing in an appropriate manner the information it is receiving.

A third group for assessing human physical performance includes a combination of the aforementioned first and second groups in the evaluation of everyday activities that involve a movement requiring power (e.g., lifting the body upwards against the force of gravity) followed by a stabilization period (e.g., achieving and maintaining an upright balance). For example, the assessment may include evaluation of a person standing up from a seated position, which is commonly known as the sit-to-stand maneuver. The sit-to-stand maneuver is one of the largest postural changes a human regularly performs and one of the maneuvers that most often results in falls in people with balance disorders.

Several instruments are commonly used to assess the condition of various aspects of hum an physical performance.

To measure force, mechanical power, or mechanical energy, instruments rely on resistance applying devices.

To assess balance/equilibrium, posturographic instruments based on dynamometric or force platforms may be employed to measure sway. Oculographic or nystagmographic instruments may be used to measure the movements of the eyes since eye movements are related to balance via vestibulo-ocular and oculo-spinal reflexes. Electromyographic instruments help detect the activity of muscles used in maintaining posture; electrogoniometers measure angles of the body, for example, of the limbs; accelerometers measure the movements of the head or other body parts; pulse-oximeters measure the percentage of hemoglobin that is saturated with oxygen; perfusion monitors, blood pressure cuffs or sensors, and electrocardiographers monitor cardiovascular function. Electroencephalographic equipment measures brain activity.

The sit-to-stand maneuver comprises measuring how fast a person can stand up from a seated position, and whether the person can perform the movement without using upper extremities to aid in the motion. The maneuver is typically scored by a trained observer who measures, with a stopwatch, the time it takes the person to perform the movement. Alternatively, as the sit-to-stand maneuver typically lasts less than one second, the test may be scored by measuring how many repetitions of the maneuver a person can perform in a specific amount of time or how long it takes the person to perform a fixed number of repetitions.

All the devices currently available on the market have major drawbacks in terms of usability and reliability. For instance, the devices currently used to assess muscular performance (such as those available from Biodex Medical Systems) are expensive, large, and heavy. In addition, such devices are not easily movable and they limit the measurement to predetermined simple motions that frequently only remotely resemble real life conditions.

In the vast majority of the currently existing devices to assess balance/equilibrium, a number of instruments are used separately. In some systems, force platforms are used simultaneously with electromyographers or accelerometers to assess the neuromuscular reactions to balance perturbations. In other systems, oculographic or nystagmographic instruments are used in conjunction with head-mounted accelerometers to better quantify the role of the vestibulo-ocular and oculo-spinal reflexes. However, kinesthetic information is also provided by the spine via the vestibulo-spinal reflex as well as by all other body joints. Until now, no apparatus or method has existed that can assess the function of spinal and body kinesthetic information on balance/equilibrium. Further, until now no device or method has existed that can measure the processing of one or more of vestibular, visual, auditory, kinesthetic, or somatosensory information in the nervous system and its effect on balance/equilibrium, or can use cardiovascular functional measurements to assess the effect of the cardiovascular system on a person's balance/equilibrium.

Manually scoring the sit-to-stand maneuver has several disadvantages. Manual scoring introduces a human factor into the measurement, since not all observers consider the same start and end conditions, and even if they do, their reaction times in starting and stopping a stopwatch may be different. Other significant disadvantages are that clinically important measurements, such as the symmetry and magnitude of the force generated by a person's extremities; the time required for the person to initiate the movement after a start command is given (reaction time); and the way the person controls sway and balance during and after the movement are all missing in manual scoring.

Devices do exist to provide quantitative measurements of the sit-to-stand maneuver. One such example is the dynamometric force platform produced by NeuroCom International, which can measure the movement time, symmetry, and magnitude of the force generated by the person's lower extremities, and to some extent the way the person controls sway and balance during the sit-to-stand maneuver. However, this device does not measure the reaction time. This device also does not measure the way the person controls sway and balance after reaching the standing position. The measurement of sway and balance after reaching the standing position is important because it gives an indication of how well the person is able to compensate and adapt to large postural changes and maintain balance. The measurement also provides clues about whether conditions such as cardiovascular pathologies may be present that account for an abnormal compensation and adaptation.

Accordingly, there is the need for an apparatus and methods that comprehensively assess human physical performance. The need for enhanced insight into human physical performance can be met with an apparatus according to the present invention which is able to control and acquire data simultaneously from instruments which may be combined with testing and analysis methods.

The testing and analysis methods of the present invention help assess the relationship between one or more of somatosensory function, for example, kinesthetic function; muscular function; neurological function; or cardiovascular function, thereby presenting a clinician with a more complete picture of the condition of the person being tested. Based upon the results, the clinician may tailor an exercise regimen to improve such physical performance, whether for enhancing muscular performance, such as increasing the competitiveness of an athlete; or for improving mobility and balance, for example, reducing the fear of falling in an elderly person.

### SUMMARY OF INVENTION

The apparatus according to the present invention comprises one or more force measuring devices to measure at least one of ground reaction forces or body sway. In embodiments, the apparatus may measure or record the body kinesthetic, for example, by an instrument such as an electrogoniometer. In other embodiments, the apparatus may comprise one or more instruments for measuring at least one of vestibular, visual, auditory, somatosensory, or cardiovascular systems. Such instruments for measuring at least one of vestibular, visual, auditory, somatosensory, or cardiovascular systems include, but are not limited to, oculographic or nystagmographic instruments; pulse-oximeters; perfusion monitors; blood pressure cuffs or sensors; heart rate monitors; electrocardiographers; electroencephalographs; instruments to measure neurological activity, respiration rate, respiration volume, oxygen consumption, metabolic energy expenditure, cellular glucose absorption, or body temperature; or combinations thereof. In additional embodiments, the apparatus may also comprise one or more of electromyographic instruments, accelerometers, or combinations thereof.

The apparatus of the present invention comprises a computing apparatus for processing the signals from the one or more force measuring devices and, if present, from the one or more instruments for measuring at least one of vestibular, visual, auditory, kinesthetic, somatosensory, or cardiovascular systems. The apparatus comprises an input apparatus for accepting inputs and an output apparatus. The output apparatus may show acquired data, may prompt the person being tested with instructions and/or request for information, or may present the results of analysis. According to the present invention, the apparatus comprises a storage apparatus to store acquired data and a control apparatus to control the force measuring device and other instruments.

The present invention is also directed to methods for assessing human physical performance. In embodiments, the methods of the present invention comprise assessing the muscular performance of an person by measuring ground reaction forces; measuring the postural balance/equilibrium of a person along with one or more of vestibular, visual, auditory, kinesthetic, somatosensory, or cardiovascular information; or measuring movements requiring power followed by stabilization.

### BRIEF DESCRIPTION OF THE DRAWING

The sole FIGURE is a schematic drawing of an embodiment of an apparatus according to the present invention.

### DETAILED DESCRIPTION OF THE INVENTION

According to the present invention, an apparatus 1 for assessing human physical performance comprises one or more force measuring devices 5 to measure at least one of ground reaction forces or body sway, as shown in the FIG. In embodiments, the one or more force measuring devices 5 may comprise one or more force platforms. U.S. Pat. No. 6,510,749 B1 to Pagnacco et al. discloses a force measuring apparatus that includes a platform which accommodates in-line and on-line stances and a base for supporting the platform. The force measuring apparatus comprises a sensor apparatus for measuring loads exerted against the platform. The force measuring apparatus of U.S. Pat. No. 6,510,749 B1 is incorporated herein by reference.

In embodiments of the apparatus according to the present invention, the apparatus 1 may also comprise one or more instruments 10 for measuring at least one of vestibular, visual, auditory, kinesthetic, somatosensory, or cardiovascular information. Such instruments 10 for measuring at least one of vestibular, visual, auditory, kinesthetic, somatosensory, or cardiovascular systems include, but are not limited to, electrogoniometers; oculographic or nystagmographic instruments; pulse-oximeters; perfusion monitors; blood pressure cuffs or sensors; heart rate monitors; electrocardiographers; electroencephalographs; instruments to measure neurological activity, respiration rate, respiration volume, oxygen consumption, metabolic energy expenditure, cellular glucose absorption, or body temperature; or combinations thereof. In embodiments, the nystagmographic instrument may comprise videonystagmography goggles, such as those disclosed in U.S. Pat. No. 6,461,297 B1 to Pagnacco et al. The goggles disclosed in U.S. Pat. No. 6,461,297 B1 to Pagnacco et al. are incorporated by reference herein. The apparatus of the present invention may also include one or more of electromyographic instruments, accelerometers, or combinations thereof.

The apparatus 1 of the present invention comprises a computing apparatus 15 having a central processing unit for processing signals from the one or more force measuring devices 5 and one or more instruments 10. The computing apparatus 15 may be a stand-alone personal computer or two or more computers that are connected through a network. The computing apparatus and architecture of U.S. Pat. No. 6,461,297 B1 to Pagnacco et al. and co-pending U.S. Ser. No. 10/397,161 are incorporated by reference herein.

The apparatus 1 comprises an input apparatus 20 for accepting inputs. The input apparatus 20 may include one or more of, for example, a keyboard, a mouse, a pointing device, scanner, camera, video camera, microphone, voice recognition architecture, or combinations thereof. The inputs may be data such as person-identifying information including, but not limited to, age, sex, address, telephone, social security number, insurance information, medications used, medical history, and the like. The inputs may comprise descriptions of the position of one or more parts of the body, including limbs, head, spine, or eyes. In embodiments, the inputs may comprise testing conditions including, but not limited to, type of footwear, stimuli applied to the person, ambient temperature, mental tasks the person may be directed to perform, the type of surface that the person is standing on, or combination thereof. The inputs may also comprise commands to operate the apparatus. The inputs may comprise measured data in response to testing methods.

The apparatus 1 also comprises an output apparatus 25. The output apparatus 25 may be a computer screen or monitor, liquid crystal display, head-mounted displays, or other display apparatus, speakers, headphones, printer, and the like. In embodiments, the output apparatus may show measured data in real time during the measurements or may replay the acquired data at a later time. In embodiments, the output apparatus may be used in conjunction with the input apparatus to prompt the person being tested, or a clinician, with instructions or requests for information, or to present stimuli to the person being tested.

The apparatus 1 comprises a storage apparatus 30 for electronic storage of the measured data and any input information. According to the present invention, the apparatus 1 also comprises a control or processing apparatus 35 to control or communicate with the force measuring device 5 and one or more instruments 10 and to record measured data in the storage apparatus 30. The force measuring device, instruments, and apparata are connected with conventional data transfer and electronic interconnections. In embodiments, wireless connection pathways or long distance connections may also be utilized. In embodiments, the control apparatus 35 does not need to be in the same location or in the same vicinity as the rest of the apparatus 1, thus making it possible to remotely control the apparatus.

In embodiments, the apparatus of the present invention may include normative data based on, but not limited to, age, sex, height, ethnicity, physical fitness (e.g., sedentary versus athletic), geographical area, or combinations thereof to use as a comparison for the person's measured quantities to better establish the muscular performance or postural balance/equilibrium capability of the person. The apparatus of the present invention may be self-contained and relatively small and easy to move, thereby allowing use to occur in a variety of environments, for example, in gyms, medical offices, athletic fields, homes, nursing homes, schools, retirement facilities, and the like. In embodiments, the various components of the apparatus can be linked via long distance connection pathways, thereby allowing the apparatus to be used remotely to administer testing in remote locations from a centralized location.

The present invention is also directed to methods for assessing human physical performance. In embodiments, methods may be implemented in the apparatus of the present invention by software, algorithms, or other digital communication. The methods of the present invention assess the relationships between muscular function and neurological function.

I. Assessment of Muscular Performance

One method according to the present invention is directed to assessing muscular performance by measuring ground reaction forces. The ground reaction forces may be used to quantify one or more of the amount of force, mechanical power, mechanical energy, acceleration, velocity, or distance traveled by the center of mass of a person or part of the person's body being tested during one or more movements. In embodiments, the movements may comprise one or more of squats, jumps, push-ups, lunges, dips, combinations thereof, or other similar movements.

In embodiments, the ground reaction forces of one or more extremities may be measured. For example, the movement of one leg may be measured by performing a jump using only one leg. If the test is repeated using the other leg, the symmetry between the two legs may also be determined. The method of the present invention is not limited to testing lower extremities. For example, the ground reaction forces generated by one or both arms may be evaluated.

To measure the ground reaction forces, a person may stand on a force measuring device. In embodiments, the force measuring device may be a force platform, such as disclosed in U.S. Pat. No. 6,510,749 B1 to Pagnacco et al. The weight of the person may be measured. The mass of the person may be calculated by dividing the weight by the acceleration of gravity. The person then performs one or more movements and the force is measured.

In embodiments, the weight of the person may be subtracted from the force measurement, thereby obtaining the force acting on the person excluding gravity. The force may then be divided by the person's mass, thereby obtaining the acceleration of the person's center of mass. By integrating the acceleration in time, the velocity may be obtained. By integrating the velocity in time, the displacement may be obtained. By multiplying the overall force by the velocity, the mechanical power may be obtained. By multiplying the overall force by the displacement, the mechanical energy may be obtained. According to the present invention, the calculations may occur in real time during the testing or may be done later after the test is concluded.

By using this method, it may be possible to quickly determine how much force or power a person may generate during a particular movement, for example, how high an athlete can jump.

The method of the present invention may also measure one or more of the force, mechanical power, mechanical energy, acceleration, velocity, or the distance traveled by objects handled by a person during one or more movements. In embodiments, a person's exact weight may be measured by the force measuring device. The person may then be given an object and one or more movements may be performed. In embodiments, the object may be, for example, a weight, dumbbell, barbell, ball, or any other suitable object.

The weight of the person may be removed from the calculations, thereby concentrating the analysis on the additional weight of the object. Accordingly, it may be possible to measure one or more of the force, power, energy, acceleration, velocity or displacement that a person can apply to an object.

In embodiments, the repetitions of the one or more movements may be measured, as the movements have a cyclic component with every repetition. Thus, the changes from repetition to repetition may be measured, thereby providing data about the level of fatigue and the person's physical performance and condition. An advantage of this method is that it allows the quantitative measurement of physical and muscular performance in a variety of real-life movements involving different parts of a person's body.

II. Assessment of Postural Balance/Equilibrium and Nervous System Processing of Kinesthetic Information

Another method according to the present invention is directed to assessing physical performance by measuring and comparing sway when a person is standing on a force measuring device and at least one of the spine or body are placed in one or more different configurations so as to produce one or more kinesthetic inputs to the nervous system. In embodiments, the force measuring device may be a force platform, such as disclosed in U.S. Pat. No. 6,510,749 B1 to Pagnacco et al. The tests may be performed on one or more stable or unstable surfaces.

According to this method, spinal configurations may include, but are not be limited to, one or more head positions, for example, head tilted up, down, to the side, turned left, right, diagonally, or combinations thereof. The configurations may also include one or more torso positions, for example, torso bent forward, backward, leftward, rightward, or combinations thereof.

Body positions may include, but are not limited to, one or more positions as to arms, feet, legs, or combinations thereof. For example, positions of the arms may include both arms raised over head; left or right arm raised over head; both arms outstretched to the front; left or right arm outstretched to the front; or combinations thereof. Feet positions may include both feet together; one foot at an angle to the other; one foot in front of the other; standing on tiptoes; or combinations thereof. In embodiments, the foot may be at an angle from about 0 up to about 180 degrees, for example about 45 degrees, with respect to the other foot. Leg positions may include knees partially bent and knees fully bent; left leg out to side; left leg out to front; left leg out to rear; left leg out at an angle to front; left leg out at an angle to rear; right leg out to side; right leg out to front; right leg out to rear; right leg out at an angle to front; right leg out at an angle to rear; or combinations thereof. In embodiments, the leg may be out at an angle in the front or to the rear from about 0 up to about 180 degrees, for example about 45 degrees, with respect to the other (standing) leg.

By comparing the sway of a person in different spinal and/or body positions, it may be possible to determine positions that produce erroneous somatosensory information. It may be possible for a trained medical professional to deduce, diagnose, and often correct the origin of the problem, for example, lower back pain, sciatica, pinched nerves, and the like.

III. Assessment of Postural Balance/Equilibrium and Nervous System Processing of at Least one of Vestibular, Visual Auditory or Somatosensory Information

Another method according to the present invention is directed to assessing physical performance by measuring sway when a person is standing on a force measuring device and the effect of processing at least one of vestibular, visual, auditory, or somatosensory systems in the nervous system.

In embodiments, the force measuring device may be a force platform, such as disclosed in U.S. Pat. No. 6,510,749 B1 to Pagnacco et al. The tests may be performed on one or more stable or unstable surfaces.

While a person is standing on the force measuring device, tests may be performed with one or more of visual stimuli, auditory stimuli, olfactory stimuli; thermic stimuli, pressure (tactile) stimuli, or combinations thereof to measure the effect on nervous system processing of such information. In embodiments, the visual stimuli may include, but are not limited to, shapes, letters, lights, flashing lights, or other visual cues moving around in the person's field of vision, while the rest of the environment is stationary. The auditory or acoustic stimuli may include, but are not limited to, having the person listen and react to sounds of various volumes, frequencies, rhythms, tones, or word utterances. The olfactory stimuli may include, but are not limited to, having the person smell and react to essences, perfumes, or smells. The thermic stimuli may include, but are not limited to, exposing the person to cold or warm temperatures, for example, by changing the room temperature or by applying cold or warm objects to localized areas of the person's body. The pressure (tactile) stimuli may include, but are not limited to, having the person feel and react to being touched in different parts of the body, to being pushed in different directions, or to getting in contact with objects or other persons.

The person may also be required to perform a task. In embodiments, the task may include, but is not limited to, using different breathing regimens, gazing at specific directions, performing various mental tasks, or combinations thereof. Examples of gazing exercises include, but are not limited to, asking the person to look up, look down, look right, look left, follow a finger, and the like. The gazing exercises may be with the eyes open or closed. Any metal task that distracts the person from concentrating on sway and activates the brain by consuming more oxygen may be sufficient. Examples of the mental tasks include, but are not limited to, counting forward or backwards; reciting the alphabet forwards or backwards; spelling words; performing simple mathematic computations (e.g., addition, subtraction, multiplication, or division); reciting poems or prose passages; word association games; and the like. For example, the person may be asked to count backwards from a number (e.g., from 100 in numbers of 7: 93, 86, 79, etc.) or may be asked to provide homonyms, synonyms, or antonyms for specific words.

By comparing the effect of one or more of visual stimuli, auditory stimuli, olfactory stimuli, thermic stimuli, pressure stimuli, breathing, gaze, or mental tasking on the sway of a person, it may be possible to infer the presence of certain pathologies affecting the processing of the information used to maintain balance by the nervous system. In embodiments, this method may also be combined with the method in Section II above to further determine how somatosensory spinal and/or body data integrate with the rest of the information used for balance.

Many of the conditions used in testing may also be used to stimulate different parts of the nervous system for therapeutic regimens (using specific exercises or chiropractic/osteopathic adjustment or manipulations). Thus, the apparatus and methods of the present invention may allow medical personnel to verify in near real time if a therapeutic regimen is having a positive beneficial effect or if it is not producing the desired effects. The clinician can not only quantify outcomes, but may also be able to immediately change tactics and employ different therapeutic techniques that may produce better results.

IV. Assessment of Postural Balance/Equilibrium and Physiological Function

Another method according to the present invention is directed to assessing physical performance by simultaneously measuring sway and at least one other physiological parameter. In embodiments, the force measuring device may be a force platform, such as disclosed in U.S. Pat. No. 6,510,749 B1 to Pagnacco et al. While standing on a force measuring device, an additional physiological parameter is measured. In embodiments, the physiological parameter includes, but is not limited to, blood pressure, blood oxygenation, accelerations, myoelectric activity, heart rate, cardiac activity, brain activity, neurological activity, respiration rate, respiration volume, oxygen consumption, metabolic energy expenditure, cellular glucose absorption, body temperature, ocular movement, nystagmographic measurements, body joint angles, or combinations thereof. The tests may be performed on one or more stable or unstable surfaces.

Analysis of the measured data helps gain insight on the overall health of the person by assessing relationships between one or more of balance, neurological function, muscular function, or cardiovascular function.

For example, a person with impaired cardiovascular function may have a balance problem and/or some neurological deficits, even if temporary and related to a specific posture. A common situation of this type is a condition called postural hypotension, in which a person's blood pressure decreases rapidly when changing posture, for example, when going from a reclining position to a sitting or standing position; when going from a supine or seated position to an upright stance; or when tilting or moving the head or the body in a specific position or direction. In embodiments, combining simultaneous recording of force-platform-measured sway and at least one of pulse-oximetry, electrocardiography, blood pressure, or blood perfusion monitoring may be used to determine the response of the cardiovascular system to postural changes and immediately detect those situations.

As another example, some individuals have episodes of short reversible neurological and balance deficits that result in temporary abnormal eye movements and body sway. These episodes may have a cardiovascular origin (e.g., conditions like arrhythmia or cardiac valves pathologies) or a neurological origin. In these situations, simultaneously measuring ocular movements as well as cardiovascular function using pulse-oximetry, electrocardiography, blood pressure monitoring and/or blood perfusion monitoring could quickly show the clinician if the origin of the problem was cardiovascular or neurological.

V. Assessment of Movement Requiring Power Followed by a Stabilization Period

Another method according to the present invention may be directed to assessing neuromuscular abilities of a person by measuring the reaction time during and the sway after a sit-to-stand maneuver.

Sway may be measured by one or more force measuring devices. In embodiments, the one or more force measuring device may comprise a force platform, such as disclosed in U.S. Pat. No. 6,510,749 B1 to Pagnacco et al. The sway may be analyzed using both transient analysis methods as well as standard posturography analysis, by comparing the person's sway after the sit-to-stand maneuver with the same person's sway in the types of spinal and/or body configurations disclosed on Section II above.

In embodiments, a person may be seated on a chair positioned near or on top of the force measuring device, with the feet resting on the force measuring device. In this configuration, it is possible to perform the same analysis as Section I above, thereby evaluating how much one or more of the force, mechanical power, or mechanical energy may be generated during the sit-to-stand maneuver as well as one or more of the acceleration, velocity, or distance traveled by the center of mass of the person during the standing phase. Moreover, if each foot is rested on a different force measuring device, the individual values generated by each leg may be assessed.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. An apparatus for assessing human physical performance, comprising:
one or more force measuring devices;
one or more instruments for measuring at least one of vestibular, visual, auditory, kinesthetic, somatosensory, or cardiovascular information;
a control apparatus for controlling the one or more force measuring devices and the one or more instruments;
a computing apparatus for processing signals from the one or more force measuring devices and the one or more instruments;
a storage apparatus;
an input apparatus; and
an output apparatus.

2. An apparatus according to claim 1, wherein the one or more force measuring devices comprises one or more force platforms.

3. An apparatus according to claim 1, wherein the one or more instruments is selected from the group consisting of an electrogoniometer, an oculographic or nystagmographic instrument; a pulse-oximeter; a perfusion monitor; blood pressure cuffs or sensors; a heart rate monitor; an electrocardiographer; an electroencephalograph; an instrument to measure neurological activity, respiration rate, respiration volume, oxygen consumption, metabolic energy expenditure, cellular glucose absorption, or body temperature; and combinations thereof.

4. An apparatus according to claim 3, further comprising at least one of an electromyographic instrument or accelerometer.

5. An apparatus according to claim 1, wherein the input apparatus comprises one or more of a keyboard, a mouse, a pointing device, scanner, camera, video camera, microphone, voice recognition architecture, or combinations thereof.

6. An apparatus according to claim 1, wherein the output apparatus comprises one or more of a computer screen or monitor, a liquid crystal display, head-mounted display, speakers, headphones, printer, or combinations thereof.

7. A method for assessing human physical performance, comprising:
having a person stand on a force measuring device; and
assessing at least one of:
(A) muscular performance;
(B) postural balance/equilibrium and nervous system processing of kinesthetic information;
(C) postural balance/equilibrium and nervous system processing of at least one of vestibular, visual, auditory, or somatosensory information;
(D) postural balance/equilibrium and at least one other physiological parameter; or
(E) neuromuscular abilities of a person during a movement requiring power followed by a stabilization period.

8. A method according to claim 7, comprising:
having a person stand on a force measuring device;
measuring ground reaction forces as the person performs one or more movements on the force measuring device; and
calculating from the ground reaction forces at least one of force, mechanical power, mechanical energy, acceleration, velocity, or distance traveled by the center of mass of the person or by an object held by the person.

9. A method according to claim 8, wherein the one or more movements comprises at least one of squats, jumps, push-ups, lunges, dips, or combinations thereof.

10. A method according to claim 8, comprising
having the person perform repetitions of the one or more movements; and
for each repetition, calculating at least one of force, mechanical power, mechanical energy, acceleration, velocity, or distance traveled by the center of mass of the person or by an object held by the person.

11. A method according to claim 8, further comprising having the person carry an object during the one or more movements.

12. A method according to claim 11, wherein the object comprises a weight, dumbbell, barbell, or ball.

13. A method according to claim 7, comprising:
having a person stand on a force measuring device; and
assessing postural balance and equilibrium and nervous system processing of at least one of spinal or body kinesthetic information by measuring sway of the person in different spinal configurations, body positions, or combinations thereof.

14. A method according to claim 13, comprising measuring the sway of the person in one or more different spinal configurations comprising one or more head positions, torso positions, or combinations thereof.

15. A method according to claim 13, comprising measuring the sway of the person in one or more different body positions comprising one or more leg positions, arm positions, feet positions, or combinations thereof.

16. A method according to claim 7, comprising:
having a person stand on a force measuring device; and
assessing postural balance and equilibrium and nervous system processing of at least one of vestibular, visual, auditory, or somatosensory information by measuring the sway of the person and the effect of at least one of visual stimuli, acoustic stimuli, olfactory stimuli, thermic stimuli, pressure stimuli, or performance of a task.

17. A method according to claim 16, comprising measuring the person's response to visual stimuli.

18. A method according to claim 16, comprising measuring the person's response to acoustic stimuli.

19. A method according to claim 16, comprising measuring the person's response to olfactory stimuli.

20. A method according to claim 16, comprising measuring the person's response to thermic stimuli.

21. A method according to claim 16, comprising measuring the person's response to pressure stimuli.

22. A method according to claim 16, wherein the task is selected from the group consisting of a breathing regimen, gazing at specific directions, performing a mental task, and combinations thereof.

23. A method according to claim 7, comprising:
having a person stand on a force measuring device; and
assessing postural balance and equilibrium and at least one other physiological parameter.

24. A method according to claim 23, comprising measuring at least one physiological parameter selected from the group consisting of heart rate, blood pressure, blood oxygenation, blood perfusion, electrocardiographic activity, electroencephalographic activity, neurological activity, respiration rate, respiration volume, oxygen consumption, metabolic energy expenditure, cellular glucose absorption, body temperature, accelerations, myoelectric activity, ocular movement, nystagmographic measurements, and combinations thereof.

25. A method according to claim 7, comprising:
assessing the neuromuscular abilities of a person by measuring reaction time of a person during one or more sit-to-stand maneuvers on a force measuring device; and
measuring sway of the person after reaching a stand position on the force measuring device.

26. A method according to claim 25, wherein the force measuring device comprises a force platform.
